(19) United States
(12) Patent Application Publication
(10) Pub. No.: US 2021/0214657 A1
(43) Pub. Date: Jul. 15, 2021

(54) Ball-Shaped Lavatory Block Comprising Two Different Compositions

(71) Applicant: Henkel AG & Co. KGaA, Duesseldorf (DE)

(72) Inventors: Thomas Holderbaum, Hilden (DE); Tobias Segler, Duesseldorf (DE); Tatiana Schymitzek, Krefeld (DE)

(21) Appl. No.: 17/214,302

(22) Filed: Mar. 26, 2021

Related U.S. Application Data
(63) Continuation of application No. PCT/EP2019/067433, filed on Jun. 28, 2019.

(30) Foreign Application Priority Data
Sep. 27, 2018 (EP) 18197350

(51) Int. Cl. C11D 17/00; C11D 17/04; C11D 1/83; C11D 1/37; C11D 3/40; C11D 3/50; E03D 9/03

(52) U.S. Cl. CPC C11D 17/0056; C11D 17/046; C11D 1/83; C11D 1/22; C11D 3/40; C11D 3/50; E03D 9/032; C11D 1/37

(57) ABSTRACT

The invention relates to a solid toilet block having at least a first composition and a second composition obtained by a non-concentric coextrusion step, and to a method for producing such a toilet block and to a device having such a toilet block.

### FIELD OF THE INVENTION

Toilet cleaning pieces, also known as toilet blocks, have long been used for cleaning, disinfecting and scenting toilets under the bowl rim (rim blocks) and in the cistern (in-tank blocks or cistern blocks). In recent years, aesthetics and performance have become increasingly important. This has led, for example, to the development of solid or gel-like or liquid scented rinsers, some of which are offered in multi-chamber containers and thus allow a cleaning agent, which is released when the toilet is flushed, to be combined with permanent room scenting.

### BACKGROUND OF THE INVENTION

Usually a rim block is formed of a single composition. The composition is extruded, the strand cut and optionally formed.

For some time now, rim blocks have also been known that have two different compositions. This makes it possible to combine different active substances, such as dyes, fragrances in the form of perfume oils, or biocides, in the toilet block. A different release profile can be generated by a toilet block having a plurality of compositions than is possible in the case of a toilet block having one composition. By using at least two compositions, a first active substance can be used in the first composition and a second active substance can be used in the second composition. If, for example, only the first active substance is compatible with the first composition, i.e., for chemical reasons or physicochemical reasons, the second active substance cannot be used in the first composition, both active substances can still be used in the toilet block by using an additional second composition that contains the second active substance.

Rim blocks are known from the prior art which have two different compositions. However, these blocks are obtained by concentric coextrusion, the strand being subsequently cut. Such toilet blocks have a shell and an inner core. No further forming step is provided. Due to the geometry of the two compositions, the shell dissolves first. This results in relatively strong release of the first active substance at the beginning of use and stronger release of the second active substance at a later point in time. As a rule, however, constant release of the two active substances is desired over substantially the entire period of use. Such coextruded rim blocks are unsuitable for this.

It was therefore desirable to provide an elegant and aesthetic toilet block that can contain different active substances and that is rinsed evenly over its entire service life and swells as little as possible.

### BRIEF SUMMARY OF THE INVENTION

The object is achieved by a toilet block according to the invention having at least a first composition and a second composition, the first composition differing from the second composition and the production of the toilet block comprising a coextrusion step, the first composition being in spatial contact with a portion of the side wall of the passage and the second composition being in spatial contact with another portion of the side wall.

In connection with the present invention, the term extrusion stands for a method in which one or more compositions are preferably continuously pressed out of an opening under pressure. The opening is also known to a person skilled in the art as a profile die. The resulting body having the cross-section of the opening is called the extrudate.

In connection with the present invention, the term coextrusion stands for an extrusion method in which at least a first composition and a second composition are brought together in an extrusion head, also called an extruder head.

The extruder head forms a passage, one end of the passage being formed by a profile die through which the brought-together compositions can emerge as extrudate.

During the extrusion, there is substantially no complete homogeneous mixing of the first composition and the second composition in the extrusion head. In other words, the first composition and the second composition are retained in that they can still be distinguished from one another in the extrudate. This does not rule out the fact that in the contact region of the first composition and the second composition there may be some slight mixing or blending of the compositions.

The passage in the extruder head has a side wall. The first composition and the second composition are brought together in the extruder head such that the first composition is in spatial contact with a portion of the side wall of the extruder head and the second composition is in spatial contact with another portion of the side wall of the extruder head.

Such an arrangement differs in particular from concentric coextrusion, in which a first composition is completely in spatial contact with the side wall of the extruder head and a second composition is extruded concentrically within the first composition, i.e., the second composition has no spatial contact with the side wall of the extruder head. In the extrudate, the second composition is thus concentrically enclosed by the first composition. The extrudate according to the invention and thus also the toilet block according to the invention thus differ significantly from such a concentric extrudate known from the prior art.

The toilet block according to the invention, the first composition having a first active substance and the second composition having a second active substance, the first active substance and the second active substance differing from one another.

Preferably, the first active substance is a first dye and the second active substance is a second dye.

The toilet block according to the invention, the first active substance being a first fragrance and the second active substance being a second fragrance.

The invention preferably comprises a toilet block and a method for producing a toilet block, the production of the toilet block comprising a forming step after the coextrusion step.

According to a preferred embodiment, the compositions form the surface of the toilet block in substantially equal proportions.

The toilet block according to the present invention preferably has at least a C₂ symmetry, i.e., the block is symmetrical in that at least a twofold axis of rotation is present as the symmetry element. The invention preferably further comprises a toilet block and a method for producing a toilet block, the toilet block being rotationally symmetrical, at least one C_{∞} axis (C axis at infinity) being present as the symmetry element, higher-symmetric geometries being of course also included (e.g., a plurality of C_{∞} axes). A preferred geometry is the cylinder, which can be rinsed evenly due to its rotationally symmetrical geometry, i.e., the water can in particular run off evenly over the cylinder shell. Such a symmetry is particularly preferred in the case of a toilet block which comprises two compositions that form the surface of the toilet block in substantially equal proportions. In accordance with the symmetry of the toilet block, both compositions are released into the flushing water simultaneously. This is in contrast to a concentric geometry in which the shell is rinsed first and then the core of the cylinder.

A spherical form is very particularly preferred because, due to its symmetrical geometry, it allows the toilet block to be rinsed substantially evenly, i.e., the water can run off evenly on all sides. Such a symmetry is particularly preferred in the case of a toilet block which comprises two compositions that form the surface of the toilet block in substantially equal proportions. In accordance with the symmetry of the toilet block, both compositions are released into the flushing water simultaneously.

The invention preferably comprises a toilet block and a method for producing a toilet block, the extrudate being formed into a sphere by the forming step and the toilet block thus having a substantially spherical geometry.

As a rule, constant release of the two active substances over substantially the entire period of use is desired. In a sphere-forming step, the two compositions are “interwoven” with one another on the surface in that the two compositions are interwoven with one another in the manner of streaks and are thus completely evenly distributed over the surface. Such a distribution of the compositions on the surface of the block can be achieved in particular by simultaneous cutting and sphere-forming of a cylindrical (non-concentric) coextrudate.

A spherical form is also particularly preferred, since this is rinsed evenly due to its highly symmetrical toilet block geometry. Rinsing water is directed evenly along the surface of the sphere and comes into contact with the entire surface of the block evenly.

The invention preferably comprises a toilet block and a method for producing a toilet block, the extrudate being cut into smaller units.

The invention preferably comprises a toilet block and a method for producing a toilet block, the extrudate being cut into smaller units of approximately the same size.

The invention preferably comprises a toilet block and a method for producing a toilet block, the passage being substantially cylindrical.

The circumference of the part cylinder of the extrudate which corresponds to the first composition can be different from the circumference of the part cylinder which corresponds to the second composition. This allows the appearance and the release profile of the toilet block to be customized. It is possible, for example, that the first composition forms a part cylinder having ¾ of the circumference of a circle and the second composition forms a part cylinder having ¼ of the circumference of the circle. It is also possible, for example, that the first composition forms a part cylinder having ⅔ of the circumference of a circle and the second composition forms a part cylinder having ⅓ of the circumference of the circle. However, it is preferred that the two compositions each form a half cylinder. In other words, if the passage is substantially cylindrical, the extrusion strand preferably has half cylinders of the same size.

In addition to a cylindrical strand geometry, other geometries are also possible, such as triangular, rectangular, star-shaped, or other figurative shapes. A further forming step can be omitted here. Such variations can advantageously be used to customize a release profile for active substances of the first composition compared with the second composition.

The invention preferably comprises a toilet block and a method for producing a toilet block, the portion of the side wall of the passage with which the first composition is in contact and the portion of the side wall of the passage with which the second composition is in contact having a surface area of substantially the same size.

If a cylinder is divided by a plane containing its axis, two congruent half cylinders are obtained. As with the cylinder, the radius and the height determine the half cylinder. If the passage is substantially cylindrical, the extrusion strand has half cylinders of the same size.

According to a further aspect, the invention relates to a device having at least one toilet block according to the invention, the at least one toilet block being in a chamber of the device, it being possible for the device to be attached to the rim of a toilet.

The device preferably has at least two, preferably at least three and more preferably at least four, toilet blocks.

A preferred device is described below by way of example. The dispensing device consists of a container which has, at its head end, an inlet opening through which the flushing water can enter the container via the flushing water distributing element. The flushing water that has entered the container dissolves some of the toilet cleaning block stored in the container, the flushing water now loaded with the corresponding preparation leaving the container via the outlet opening and thus being released into the interior of the toilet bowl.

In the embodiment described here, the flushing water distributing element is shaped like a plate. In addition, however, trough-like, slide-like or ramp-like shapes and any combinations thereof are also possible, for example. The flushing water distributing element is in the flushing water stream in the toilet bowl, the main flow direction of the flushing water stream usually being directed downward in the direction of gravity. The flushing water stream is broken up by the flushing water distributing element, which acts similarly to a deflector plate, and is distributed over the surface of the flushing water distributing element. The surface of the flushing water distributing element facing the flow of flushing water may comprise liquid-guiding and/or liquid-distributing structures, such as transversely and/or longitudinally extending grooves, capillaries or gratings.

The flushing water distributing element may also comprise an opening through which flushing water can flow into the inlet opening in the container.

The toilet basket disclosed has a holding element by means of which the toilet basket can be detachably attached to the rim of a toilet bowl by a user.

The holder comprises a first spring element and a second spring element, the first spring element having a substantially vertical spring travel and the second spring element having a substantially horizontal spring travel when the toilet rinser is installed in the toilet, meaning that the toilet basket can be secured to toilets which have different bowl rim thicknesses and designs in an improved and a more flexible manner.

According to a further embodiment, the dispensing device has a plate-like flushing water distributing element which, when the dispensing device is installed in a toilet bowl, extends directly from below the inlet opening of the container toward the toilet rim.

According to a further embodiment, the passage has, on the inside, a cross-sectional area which is preferably approximately 1 to 10 cm² and more preferably 2 to 5 cm².

According to a further embodiment, the passage has, on the inside, a diameter that is preferably between 15 mm and 30 mm, more preferably between 15 and 25 mm and even more preferably between 18 mm and 22 mm.

If the toilet block is formed into a sphere, the sphere volume is preferably 2 cm³ to 20 cm³, more preferably 5 cm³ to 15 cm³ and even more preferably 8 cm³ to 9 cm³.

According to a further embodiment, the sphere diameter is preferably 10 mm to 40 mm, more preferably 15 mm to 30 mm and even more preferably 22 mm to 28 mm.

According to a further embodiment, the sphere weight is preferably 5 to 30 g, more preferably 5 to 20 g and even more preferably 10 to 15 g.

According to a further aspect, the invention relates to a method for producing a solid toilet block, comprising a coextrusion step, the at least a first composition and a second composition, before exiting through a profile die, being brought together in a passage of an extrusion head such that the first composition is in spatial contact with a portion of the side wall of the passage and the second composition is in spatial contact with another portion of the side wall.

According to a preferred embodiment, the method is designed in such a way that the method further comprises: at least one cutting step, the extrudate being cut into smaller units by the forming step.

According to a further preferred embodiment, the method is designed in such a way that the method further comprises: at least one cutting step, the extrudate being cut into smaller units by the forming step. The cutting step preferably takes place substantially simultaneously with a forming step, the forming step preferably being a sphere-forming step.

According to a preferred embodiment, the method is designed in such a way that the method further comprises: a forming step, the extrudate being formed into a sphere by the forming step and the toilet block thus having a substantially spherical geometry.

The strand is preferably cut to dimensions that are similar in all three spatial directions. Subsequent ball forming or sphere forming is possible, but not absolutely necessary.

In addition to a cylindrical strand geometry, other geometries are also possible, such as triangular, rectangular, star-shaped, or other figurative shapes. It is advantageous here that a further forming step is omitted. Furthermore, a variation of the release profile can also be achieved in this way.

According to a preferred embodiment, the method is designed in such a way that the cutting step and the forming step are carried out simultaneously. For example, the strand is cut by rotating rollers and simultaneously formed into a sphere by the roller shape. Such methods are known for extrudates having a single composition from the production of chocolate spheres. In the case of the present invention, it was surprisingly found that such methods, in the case of extrudates having two compositions, interweave the two compositions with one another in the manner of streaks.

The following examples show possible compositions V1, V2, V3, V4 and V5 by way of example. All quantities are in wt. %:

| | V1 | V2 | V3 | V4 | V5 |
|---|---|---|---|---|---|
| C₁₀₋₁₃ lin. | 26 | — | 12.4 | 21 | — |
| alkylbenzene | | | | | |
| sulfonate-Na | | | | | |
| Fatty alcohol | — | 7.4 | — | — | — |
| sulfate-Na | | | | | |
| C₁₂ fatty alcohol | — | 17.4 | 12.4 | — | — |
| sulfate-Na | | | | | |
| C₁₄₋₁₆ olefin | 18 | — | — | 23 | 20 |
| sulfonate-Na | | | | | |
| C₁₆₋₁₈ fatty alcohol | 8 | 17 | 17 | — | 17 |
| ethoxylate 25 EO | | | | | |
| Cellulose | — | 3 | — | — | — |
| Trisodium citrate | — | 2 | 2 | 0.3 | 2 |
| dihydrate | | | | | |
| Sodium sulfate | to make | to make | to make | to make | to make |
| | up | up | up | up | up |
| | to 100 | to 100 | to 100 | to 100 | to 100 |
| Sodium carbonate | — | 0.95 | 0.95 | — | 0.95 |
| C₁₂₋₁₈ fatty acid | — | 8 | 15 | — | 15 |
| monoethanolamide | | | | | |
| Sodium silicate | — | — | — | 3 | — |

By now, spherical toilet blocks with a wide variety of combinations of different surfactants have become known. For example, the compositions can contain only anionic surfactants, only non-ionic surfactants or mixtures thereof. Cationic surfactants are also conceivable. The compositions listed are therefore only to be understood as examples and not as being restrictive.

A first composition and a second composition are selected from V1, V2, V3, V4 and V5. Furthermore, active substances are added to the first composition and the second composition. F1 is a yellow dye and F2 is a blue dye. P1 is a lemon fragrance and P2 is a pine fragrance. By varying the compositions and the active substances, the following combinations K1 to K5 result:

| | Composition | Active | Composition | Active |
|---|---|---|---|---|
| | 1 | substance | 2 | substance |
| Combination K1 | V1 | F1 | V1 | F1 |
| Combination K2 | V1 | F1, P1 | V4 | F2, P2 |
| Combination K3 | V2 | F1, P1 | V3 | F2 |
| Combination K4 | V2 | F2, P2 | V4 | F1, P2 |
| Combination K5 | V2 | F2, P1 | V5 | F1, P1 |
| Combination K6 | V3 | P1 | V4 | P2 |

A solid toilet block is formed therefrom, the production of the toilet block comprising, according to the invention, a coextrusion step, the first composition being in spatial contact with a portion of the side wall of a passage of the extruder and the second composition being in spatial contact with another portion of the sidewall.

The passage of the extruder is substantially cylindrical. The compositions are used in substantially the same proportions, i.e., the cylindrical extrudate has two partial volumes of approximately the same size. The compositions therefore each form a half cylinder, i.e., the cylindrical extrusion strand is formed by two half cylinders of the same size.

After extrusion, the blocks were subjected to a cutting step and then to a forming step, the extrudate being formed into a sphere by the forming step and the toilet block thus having a substantially spherical geometry. Using the toilet blocks obtained, a device was obtained having four chambers, each chamber being filled with one of the round toilet blocks. The blocks had a clearly visible and evenly structured marbling, i.e., the two compositions were interwoven with one another in the manner of streaks and took up areas of approximately the same size on the surface of the toilet block. Such a toilet block is shown in FIG. 5. In contrast to known spherical blocks, which can be obtained by concentric coextrusion and thus have a core-shell geometry, the blocks obtained here allow uniform release of both compositions into the water, which can flow evenly down over the round spherical surface.

The device is attached to the rim of a toilet and is tested by flushing, it being possible to observe substantially uniform flushing of the two compositions and thus of the different active substances in all toilet blocks.

### BRIEF DESCRIPTION OF THE DRAWINGS

The suitable toilet cleaning blocks according to the invention are explained with reference to the accompanying drawings. In the drawings, in detail:

FIG. 1 shows concentric coextrusion from the prior art;

FIG. 2 shows non-concentric coextrusion according to the invention;

FIG. 3 shows a device for non-concentric coextrusion according to the invention;

FIG. 4 shows a toilet block according to the invention; and

FIG. 5 shows another toilet block according to the invention.

### LIST OF REFERENCE SIGNS

105 extruder head
110 passage of the extruder head
110a side wall of the extruder head
121 first composition
122 second composition
205 extruder head
210 passage of the extruder head
210a part of the side wall of the extruder head
220b part of the side wall of the extruder head
221 first composition
222 second composition
300 device for producing the toilet block according to the invention
305 passage of the extruder head
310 passage of the extruder head
310a portion of the side wall of the extruder head
310b portion of the side wall of the extruder head
312 profile die
320b side wall of the extruder head
321 first composition
322 second composition
330 interface
402 toilet block obtained without a sphere-forming step
521 first composition
522 second composition
502 toilet block obtained with a sphere-forming step
521 first composition
522 second composition

### DETAILED DESCRIPTION OF THE INVENTION

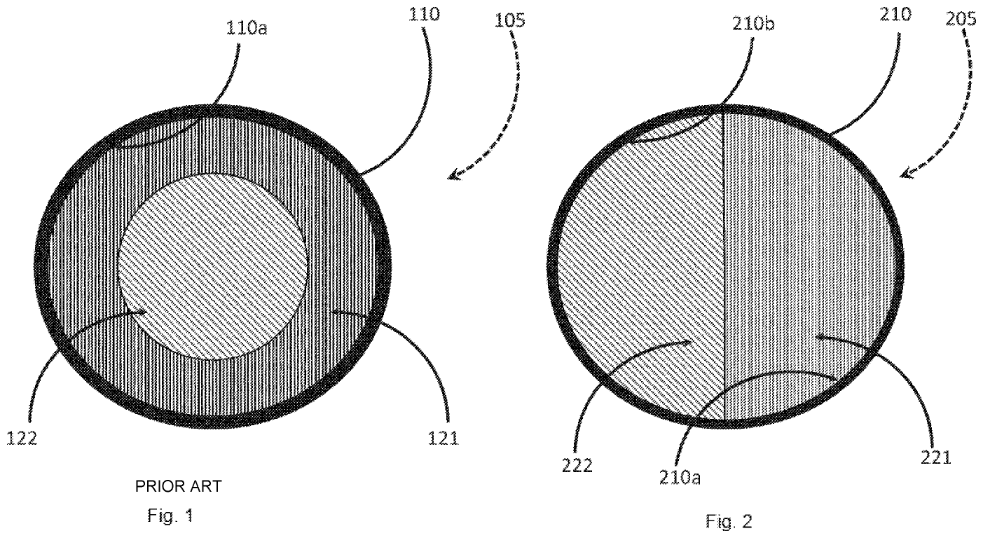

FIG. 1 shows an extruder head (105) having a passage (110), which head is used in a method to produce a solid toilet block according to the prior art. The extruder head provides an extrudate having at least a first composition (121) and a second composition (122). The first composition and the second composition differ from one another. The production of the toilet block comprises a coextrusion step that takes place concentrically. In concentric extrusion, the first composition is in spatial contact with the entire side wall of the passage (110a), while the second composition is not in spatial contact with the side wall of the passage.

FIG. 2 shows an extruder head (205) having a cylindrical passage (210), which head is used in a method to produce a solid toilet block according to the invention. The extruder head provides an extrudate having at least a first composition (221) and a second composition (222). The first composition and the second composition differ from one another. The production of the toilet block comprises a coextrusion step that takes place non-concentrically. In this extrusion according to the invention, the first composition is in spatial contact with a portion of the side wall of the passage (210a), while the second composition is in contact with another portion of the side wall (210b) of the cylindrical passage.

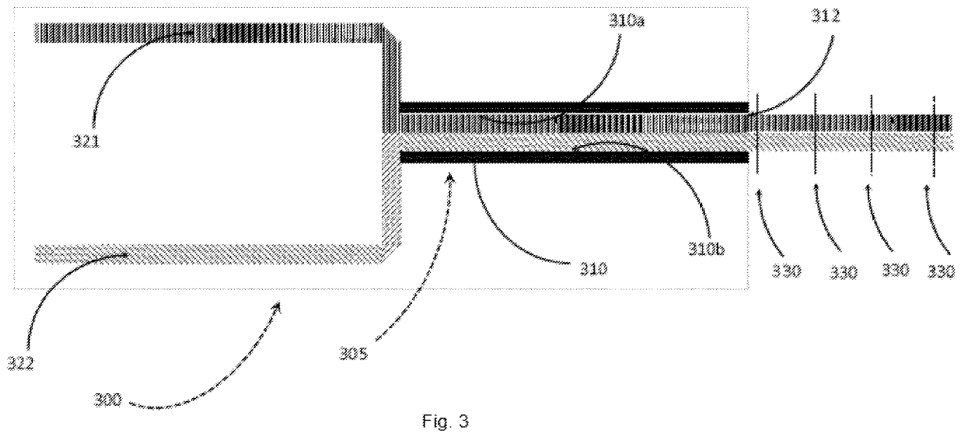

FIG. 3 shows a device (300) comprising an extruder head (305) having a passage (310), which device is used in a method to produce a solid toilet block according to the invention. The extruder head provides an extrudate having at least a first composition (321) and a second composition (322). The first composition and the second composition differ from one another. The production of the toilet block comprises a coextrusion step that takes place non-concentrically. In this extrusion according to the invention, the first composition is in spatial contact with a portion of the side wall of the passage (310a), while the second composition is in contact with another portion of the side wall (310b) of the passage. The initially separate strands are brought together in the extruder head. The coextruded strand is pressed out of the extruder head through a profile die (312). One or more cutting steps follow. According to the embodiment shown, the method is designed in such a way that the block is separated into smaller units by a plurality of cuts. The cuts can be made simultaneously or at different times.

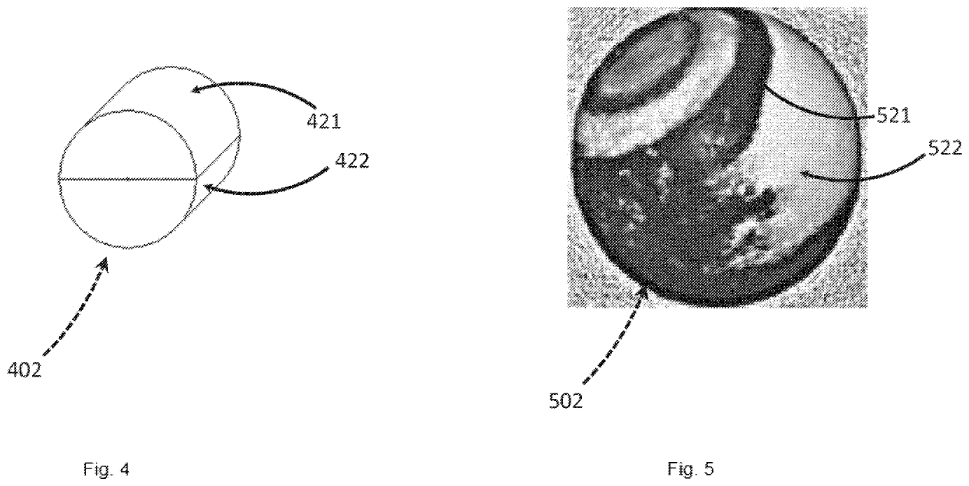

FIG. 4 shows a toilet block (402) according to the invention which was produced by means of a coextrusion step that takes place non-concentrically. In the specific embodiment shown, the toilet block consists of two half cylinders, one half cylinder consisting of a first composition (421) and the other half cylinder consisting of a second composition (422). The toilet block shown in FIG. 4 was obtained without a subsequent sphere-forming step, i.e., the cylindrical extrudate was merely cut and thus divided into smaller pieces.

FIG. 5 shows a toilet block (502) according to the invention which was produced by means of a coextrusion step that takes place non-concentrically. In the embodiment shown, the extrudate, which consists of two half cylinders, one half cylinder consisting of a first composition (521) and the other half cylinder consisting of a second composition (522), was cut into smaller pieces and formed by means of a sphere-forming step, i.e., the cylindrical extrudate was cut and formed into a sphere. In a sphere-forming step, the two compositions are “interwoven” with one another on the surface in that the two compositions are blended together in the manner of streaks and therefore occupy approximately the same area of the surface of the toilet block. Such a distribution of the compositions on the surface of the block can be achieved in particular by simultaneous cutting and sphere-forming of a cylindrical (non-concentric) coextrudate. The cylindrical coextrudate is positioned between two rotating rollers that “cut” the elongated strand into the individual toilet blocks and simultaneously form each block into a sphere. Such methods are known from the field of the production of chocolate spheres. The manufacturer of corresponding machines, known as rolling machines, for chocolate spheres is, for example, the company Krüger & Salecker Maschinenbau GmbH & Co. KG, with the ARM 0252-0601 type being particularly preferred.

What is claimed is:

1. A solid toilet block having at least a first composition and a second composition, wherein the first composition is different from the second composition and the production of the toilet block comprises a coextrusion step, wherein the first composition is in spatial contact with a portion of the side wall of the passage and the second composition is in spatial contact with another portion of the side wall, wherein the extrudate is formed by the forming step to produce a sphere and the toilet block thus has a substantially spherical geometry.

2. The solid toilet block according to claim 1, wherein the first composition has at least a first active substance and the second composition has at least a second active substance, wherein the first active substance and the second active substance differ from one another.

3. The solid toilet block according to claim 2, wherein the first active substance is a first dye and the second active substance is a second dye.

4. The solid toilet block according to claim 2, wherein the first active substance is a first fragrance and the second active substance is a second fragrance.

5. The solid toilet block according to claim 1, wherein the production of the toilet block comprises a forming step after the coextrusion step.

6. The solid toilet block according to claim 1, wherein the extrudate is cut into smaller units.

7. The solid toilet block according to claim 1, wherein the passage is substantially cylindrical.

8. The solid toilet block according to claim 1, wherein the portion of the side wall of the passage with which the first composition is in contact and the portion of the side wall of the passage with which the second composition is in contact have a substantially equal surface area.

9. A device comprising at least one toilet block according to claim 1, wherein the at least one toilet block is present in a chamber of the device, wherein the device can be attached to the edge of a toilet.

10. The device according to claim 9, wherein the device has at least two toilet blocks.

11. A method for producing a solid toilet block, comprising
a coextrusion step, wherein the at least a first composition and a second composition, before exiting through a profile die, are brought together in a passage of an extrusion head such that the first composition is in spatial contact with a portion of the side wall of the passage and the second composition is in spatial contact with another portion of the side wall, and wherein the method further comprises
a cutting step, wherein the extrudate is cut into smaller units by the cutting step, and wherein the method further comprises
a forming step, wherein the extrudate is formed into a sphere by the forming step and the toilet block thus has a substantially spherical geometry.

12. The method according to claim 11, wherein the forming step and the cutting step take place simultaneously.

13. The device according to claim 10, wherein the device has at least three toilet blocks.

14. The device according to claim 10, wherein the device has at least four toilet blocks.
